(19) United States
(12) Patent Application Publication
(10) Pub. No.: US 2015/0174055 A1
(43) Pub. Date: Jun. 25, 2015

(54) HAIR-CARE PRODUCT

(71) Applicant: Sansho Kaken Kabushiki Kaisha, Osaka-shi, Osaka (JP)

(72) Inventor: Hibiki Tsutsumi, Osaka (JP)

(21) Appl. No.: 14/414,187

(22) PCT Filed: Jul. 8, 2013
(86) PCT No.: PCT/JP2013/068568

§ 371 Date: Jan. 12, 2015

(30) Foreign Application Priority Data
Jul. 10, 2012 (JP) 2012-154335
Jan. 26, 2013 (JP) 2013-012721

(51) Int. Cl. A61K 8/92; A61Q 5/00; C11C 5/00; A61K 8/02

(52) U.S. Cl. CPC A61K 8/922; A61K 8/0204; A61Q 5/00; C11C 5/002; C11C 5/008; C11C 5/006; A61K 2800/87; A61K 2800/805

(57) ABSTRACT

The purpose of the present invention is to provide a heretofore-unknown hair-care product that is unique both in terms of performance and originality; in particular, a candle type or incense-burner-shaped hair-care product. The hair-care product is used as follows: a composition present as a solid at room temperature is heated and melted directly or indirectly by using fire, and the composition, thus fluidized, is applied to hair. This hair-care product is characterized in that the aforementioned composition comprises the composition (1) containing theobroma grandiflorum seed butter as a main component.

### TECHNICAL FIELD

The present invention relates to a hair-care product which is used as follows: a composition consisting of specific main components, said composition being present as a solid at room temperature, is heated and melted directly or indirectly by using “fire”, and the composition, thus fluidized, is applied to hair. As described herein, “fire” means “the one emitting heat and light accompanied with combustion (association with oxygen), irrespective of having or not having a flame.”

### BACKGROUND ART

### [Candle Type Cosmetics]

As a hand cream cosmetic or a cosmetic for bath (body cleansing) or body works (for human body), a candle type (that is, candle) cosmetic is known. For such cosmetics, in many cases efforts are particularly made on a scent component to emphasize an atmosphere created by candle light.

According to such type of cosmetics, not only a molten liquid at 40° C. or so obtained by melting a candle itself by utilizing the heat of a lamp can be applied to hands or a body but also “scent” can be enjoyed. Namely, in addition to the intrinsic effect as a cosmetic, it is expected that candle light and unique “scent” emitted from the molten candle liquid contribute to give a relaxed feeling to both body and soul.

### [Structure of Hair]

With regard to hair care, in “Science of hair care, Popular Science, Author: Kao Life Science Laboratory, Publisher: Yoshino Tatsuji, 2^{nd} edition published on Feb. 25, 1994”, basic features of hair are described as follows.

First, a hair can be divided into a “hair shaft” protruding from skin and a “hair root” present inside skin.

The aforementioned “hair shaft” consists of cuticle (hair cuticle) on the outside, cortex (hair cortex) on the inside, and medulla (hair medulla) on the inside, and their constitutional ratio is about 15%, about 82%, and about 3% in order.

Among the constitutional layers of a hair shaft, the cuticle has a scale-like shape and it is overlapped in a slightly staggered manner in the direction of from a root to an end of hair. On pages 19, 46, 128, 131, and 151 of the aforementioned “Science of hair care”, photographic images observed under a microscope indicating the state of cuticle are shown. Thus, it is possible to understand the microscopic state of a hair surface, that is, level of damages present on a hair.

[Cosmetics Using Theobroma grandiflorum Seed Butter]

Theobroma grandiflorum seed butter is a fat obtained from a seed of a fruit named cupuass, which is a kind of cacao naturally found in South America.

According to an internet search, it is described that this fat (theobroma grandiflorum) has an activity of protecting skin, an activity of an emollient, and an activity of absorbing ultraviolet ray, and it is also described that cosmetics having this fat blended in cleansing milk, day cream, night cream, or the like are commercially available.

Meanwhile, with regard to the Japanese signage of this fat, in many cases, “grandi” is also described as “ [symbol] (gurandi)” or “florum” is described as “ [symbol] (furoramu)” or “ [symbol] (foramu)” depending on the literatures. Furthermore, those expressions are sometimes mixedly used even in one literature.

In the present specification, it is described or translated as “ [symbol] [symbol] (theobroma grandiflorum seed butter)” both for explaining the present invention and for explaining the descriptions of the documents.

### (Non-Patent Document 1)

According to the special report under the title of “Prospect of Cosmetics and Pharmaceuticals” of “FRAGRANCE JOURNAL 2003-5, pages 73 to 77” (Non-Patent Document 1), the following explanations are given in Table 3 of page 74.

Product name: Cropure cupuass

Composition: Fruit oil of theobroma grandiflorum

Characteristics: High water-holding property

### (Non-Patent Document 2)

At pages 81 and 82 of “Introduction of formulation examples of raw materials handled by each company” in “FRAGRANCE JOURNAL 2004-6, pages 76 to 85” (Non-Patent Document 2), a formulation example of an eye care (eye rim care) cream by Croda Japan KK is described, in which “cropure cupuass” is described as a fat obtained by adsorbing and purifying, by its own technologies, fats from seeds of a cupuass fruit that is harvested in Amazon and “cropure cupuass” is described to have an effect of an emollient due to a water-holding property of phytosterol as a component contained in “cropure cupuass”. There is also described an example in which “theobroma grandiflorum seed butter (cropure cupuass)” is used as one component of A in specific formulations (mixing A to D).

### (Non-Patent Document 3)

In Table 4 of “FRAGRANCE JOURNAL, 2007, Extra Edition” (Non-Patent Document 3), an example in which “cropure cupuass (theobroma grandiflorum seed butter)” is used as one component of a formulation for anti-aging cream is described.

[Patent Document relating to cosmetics using theobroma grandiflorum seed butter]

As for the cosmetics (including hair cosmetics) in which theobroma grandiflorum seed butter is used, the following patent applications have been filed, for example.

### (Patent Document 1)

Japanese patent application laid-open (JP-A) No. 2003-206213 (Patent Document 1) discloses a cosmetic composition with high water-holding property characterized in that a cosmetic consisting of a mixture of branched fatty acid (C8 to C20) ester of sucrose, fats, and esters of fatty acid contains water.

This cosmetic has a purpose of providing a feeling like lanolin when it is used for a skin care cosmetic, a hair care cosmetic, a make-up cosmetic, or the like.

As an example of fats, theobroma grandiflorum seed butter is described (claim 4), and in Table-1 (Example) and Table-2 (Comparative Example) that are related to a formulation of a lip gloss (a shining (gloss) agent for lips), Table-3 (Example) and Table-4 (Comparative Example) that are related to a formulation of a hair cream, and Table-5 (Example) that is related to a formulation of emollient cream, formulations using theobroma grandiflorum seed butter are included (meanwhile, the lip gloss is a “cosmetic used for mucose membrane” and the hair care is a “cosmetic to be rinsed out”, and as being differently categorized by the rule in the industry of cosmetics, they do not belong to the same category).

### (Patent Document 2)

JP-A No. 2004-51517 (Patent Document 2) discloses a cosmetic with high water-holding property characterized in that a cosmetic consisting of a mixture of branched fatty acid (C8 to C20) ester of sucrose (for example, isostearic acid ester), fats, and esters of fatty acid contains water.

The invention of this document relates to anti-perspiration according to blending in a solid, stick cosmetic (see, Paragraph [0001] thereof).

Examples of the fats include various fats such as shea butter, theobroma grandiflorum seed butter, or cacao butter (see, claim 4 and Paragraph [0006] thereof).

Example 1 relates to a cream foundation and Example 2 relates to a lipstick. Meanwhile, there is no example using theobroma grandiflorum seed butter in the section of Examples.

### (Patent Document 3)

In claim 2 of JP-A No. 2010-70533 (Patent Document 3), a cosmetic for hair containing an acrylic emulsion (a), waxes (b), a carboxyvinyl polymer (c), and theobroma grandiflorum seed butter (d) is disclosed.

Furthermore, in Paragraphs [0026] to [0027] and [0041] and Example of Table 4 thereof, a mention is made that theobroma grandiflorum seed butter has an excellent water-holding property and also an addition amount of theobroma grandiflorum seed butter in the cosmetic for hair is described (according to the formulation of Table 4, it is blended at 0.5 to 8.0% by weight).

### (Patent Document 4)

In claim 3 of JP-A No. 2010-70534 (Patent Document 4), a cosmetic for hair containing additionally a liquid oil (D) or theobroma grandiflorum seed butter (E) in the cosmetic for hair of claim 1 which contains specific silicone wax (A), silicone-free wax (B), and water (C) is disclosed.

According to claim 8, the content of the component (E) relative to 100% by weight of the cosmetic for hair is 0.5 to 5% by weight, and the ratio is also 0.5 to 5% by weight in Example of Table 5.

### (Patent Document 5)

In claim 6 of JP-A No. 2010-254583 (Patent Document 5), a cosmetic for hair containing additionally theobroma grandiflorum seed butter in the cosmetic for hair of claim 1, which is characterized by containing hydrogenated castor oil derivatives including higher fatty acid residues, is disclosed.

According to the description of paragraph [0031], the content of theobroma grandiflorum seed butter relative to 100% by weight of the cosmetic for hair is 0.5 to 5% by weight. In Tables 1 to 4 that are related to Examples, it is 1% by weight for all cases, and it is blended at 0.5 to 5% by weight in Table 5.

Formulation type of the cosmetic for hair includes hair wax, hair milk, hair cream, rinse-free treatment, and hair spray, according to the descriptions of Paragraph [0042].

### (Patent Document 6)

JP-A No. 2011-57624 (Patent Document 6) discloses a cosmetic composition for hair containing 50 to 99.99% by mass of at least one oil (component A) selected from silicone derivatives and volatile hydrocarbons and 0.005 to 4% by mass of at least one oil with low melting temperature (component B) selected from Anacardiaceae mango seed oil, Sterculiaceae cupuass seed butter, Arecaceae babassu oil, and Dipterocarpaceae Shorea stenoptera butter.

The invention of Patent Document 6 relates to a cosmetic composition for hair which is suitable for hair care, in particular for damaged hair with problems that are caused by a chemical treatment or sunlight (see, Paragraph [0001] thereof).

In Paragraph [0019] thereof, there is an explanation that “Sterculiaceae cupuass seed butter is a fat also referred to as theobroma grandiflorum, and examples of a commercially available product include cropure cupuass by Croda Japan KK.”

In Paragraph [0037] thereof, there is a description that “Formulation type of the cosmetic composition for hair is not particularly limited, and examples include a liquid phase like hair oil, a gel phase like hair gel, a foam phase like hair foam, and an emulsion like hair cream.”

In Example 2 among Examples 1 to 16 (hair oil) of Table 1 thereof, an example of blending 1% by mass of cupuass seed butter is described.

### (Patent Document 7)

JP-A No. 2010-185015 (Patent Document 7) discloses a “candle having a candle main part molded by using combustible waxy materials in which a wick for drawing and burning the waxy materials melted by heating is fixed in an embedded state, characterized in that the candle main body is molded by using waxy materials containing 20 to 60% by weight of Japanese wax derived from Succedanea fruit, 10 to 30% by weight of fatty acids, and 20 to 60% by weight of paraffin”.

In Patent Document 7, explanations as follows are made; a candle can be broadly classified into a “Japanese style candle” and a “western style candle” (Paragraph [0003]); the structure of a wick of a Japanese style candle (a hollow wick obtained by wrapping Japanese paper around an axis of barrel-shaped rush (Juncus effusus Linne)) and a method for producing the Japanese style candle by repeated coating of Japanese wax on the wick are described, and also the Japanese style candle is characterized in that it has significantly less oil smoke and soot generated by combustion compared to a western style candle (Paragraph [0004]); and there are problems about a wick for a Japanese style candle in terms of production process and cost (Paragraph [0005]); or the like.

### CITATION LIST

### Non-Patent Document

Non-Patent Document 1: FRAGRANCE JOURNAL 2003-5, pages 73 to 77
Non-Patent Document 2: FRAGRANCE JOURNAL 2004-6, pages 76 to 85
Non-Patent Document 3: FRAGRANCE JOURNAL, 2007, Extra Edition

### Patent Document

Patent Document 1: JP-A No. 2003-206213
Patent Document 2: JP-A No. 2004-51517
Patent Document 3: JP-A No. 2010-70533
Patent Document 4: JP-A No. 2010-70534
Patent Document 5: JP-A No. 2010-254583
Patent Document 6: JP-A No. 2011-57624
Patent Document 7: JP-A No. 2010-185015

### DISCLOSURE OF THE INVENTION

### Problem to be Solved by the Invention

In the aforementioned Non-Patent Documents 1 to 3 and Patent Documents 1 to 6, use of theobroma grandiflorum seed butter as one component of a cosmetic is described.

However, in any one of Non-Patent Documents 1 to 3 and Patent Documents 1 to 6, a description about using a composition containing theobroma grandiflorum seed butter for the purpose of hair care after fluidization by direct or indirect heating and melting by using fire is not included.

Further, apart from the purpose of hair care, a cosmetic using a composition in which theobroma grandiflorum seed butter is contained as a “main component” (that is, a composition in which the ratio of theobroma grandiflorum seed butter is as high as 60% by weight or more, 70% by weight or more, or 80% by weight or more) is not described in any one of those documents.

Meanwhile, Patent Document 7 relates to “Japanese style candle and wick used therefor (Japanese wick)”, which is characterized in that Japanese wax derived from Succedanea fruit is used. However, there is no description about theobroma grandiflorum seed butter. Furthermore, there is also no description about cosmetics.

### Purpose of the Invention

Under the circumstances, an object of the present invention is to provide a heretofore-unknown hair-care product that is unique both in terms of performance and originality (that is, to provide a hair-care product which is used as follows: a specific composition is heated and melted directly or indirectly by using fire, and the composition, thus fluidized, is applied to hair).

### Means for Solving Problem

A hair-care product of the present invention is characterized in that;

it is a hair-care product which is used as follows: a composition present as a solid at room temperature is heated and melted directly or indirectly by using fire, and the composition, thus fluidized, is applied to hair, and

the aforementioned composition consists of a composition (1) which contains theobroma grandiflorum seed butter as a main component.

A candle type hair-care product of the present invention is characterized in that:

it consists of a composition (1) which is present as a solid at room temperature and contains theobroma grandiflorum seed butter as a main component, a wick (2) set in the composition (1), and a container (3) for accommodating the composition (1) attached with the wick (2), and

the composition (1) attached with the wick (2) is either filled or fillable in the container (3) and a region surrounding the wick (2) in the container (3) is melted and fluidized by heat generated at the time of lighting the wick (2).

A hair-care product of the present invention, which is an indirect heating type using fire, is characterized in that it consists of:

a composition (1) which is present as a solid at room temperature and contains theobroma grandiflorum seed butter as a main component,

a container (3) for accommodating the composition (1),

a heating means (4) for melting or fluidizing the composition (1) within the container (3) by heating externally the container (3) accommodating the composition (1) with fire, and

a housing (5) with an incense burner shape for accommodating each of those members.

### Effect of the Invention

### (Hair-Care Product)

The hair-care product of the present invention is a hair-care product which is used as follows: a composition which is present as a solid at room temperature is heated and melted directly or indirectly by using fire, and the composition, thus fluidized, is applied to hair.

Furthermore, the hair-care product consists of the composition (1) which contains theobroma grandiflorum seed butter as a main component.

The ratio of theobroma grandiflorum seed butter in the composition (1) is preferably high blending ratio like 60% by weight or more. More preferably, it is 70% by weight or more. Particularly preferred range is 80% by weight or more.

When the ratio is less than 60% by weight, less favorable results are obtained compared to the aforementioned high blend product in terms of hair care performance, fluidity at the time of heating and melting directly or indirectly by using fire, and use feel or the like.

The melting point of the composition (1) which is present as a solid at room temperature and contains theobroma grandiflorum seed butter as a main component is preferably set in the range of 35 to 65° C. When the ratio of theobroma grandiflorum seed butter is within the aforementioned range, the requirement of having the melting point within the range of 35 to 65° C. can be easily satisfied.

When the composition (1) is used for hair care, it is fluidized according to heating and melting directly or indirectly by using fire and an operation of transferring the melted liquid applied on fingers or palm to hair is performed. Because a temperature excessively higher than the melting temperature in the range of 35 to 65° C. is not necessary, there is no strong hot feeling. Further, as it is in a fluidized state, transfer onto hair is smoothly achieved.

Furthermore, having theobroma grandiflorum seed butter as a main component, the composition (1) transferred onto hair can smoothen, in particular, cuticle as a surface layer of hair, contributes to maintain the smooth state for a long period time, and also has an effect of suppressing hair mess by organizing the hair. As such, a desirable hair care can be achieved. The hair-care product having theobroma grandiflorum seed butter as a main component is an optimum component for exhibiting such activities.

Meanwhile, theobroma grandiflorum seed butter as a main component of the composition (1) is a component also having a water-holding property. Further, other components which may be a constitutional component of the composition (1) (for example, water-holding oil like olive oil and water-holding wax like lanolin) are also a component with a water-holding property, and they have good compatibility (fusion) with theobroma grandiflorum seed butter. Further, having a water-holding property means that it can be easily used even when it is applied to wet hair.

### (Candle Type Hair-Care Product)

As a representative example of the hair-care product, there is a candle type hair-care product. The candle type hair-care product is a hair-care product of the type in which a composition present as a solid at room temperature is “fluidized by direct or indirect heating and melting by using fire”.

The candle type hair-care product consists of the composition (1) which is present as a solid at room temperature and contains theobroma grandiflorum seed butter as a main component, the wick (2) set in the composition (1), and the container (3) for accommodating the composition (1) attached with the wick (2).

The composition (1) is either filled or fillable in the container (3) and also a region surrounding the wick (2) in the container (3) is melted and fluidized by heat which is generated at the time of lighting the wick (2).

As for the product form, there can be a form in which the solid composition (1) attached with the wick (2) is filled in the container (3) or a set form consisting of the container (3) and the solid composition (1) attached with the wick (2). For any form of the former and the latter, it is also possible that the solid composition (1) attached with the wick (2) is added as a refill or separately sold.

Because the composition (1) is either filled or fillable in the container (3), the region surrounding the wick (2) in the container (3) is melted by the heat generated by lighting of the wick (2).

Thus, by performing an operation of tilting the container (3) to supply the melted liquid from a spout (3p) to fingers or palm, and transferring it onto hair, the transfer onto the hair is smoothly achieved.

As long as the spirit of the present invention is not impaired, the container (3) can have any shape, any material, and any depth.

In that case, when the container (3) is made of ceramics, has a deep bottom, and allows leak of light through a container wall at the time of lighting, the effect of the present invention is exhibited at the highest level.

To allow the leak of light through a container wall at the time of lighting even when the container (3) is made of ceramics, for example, the thickness of the container wall is prepared to be thin, it is made of porcelain (in particular, white porcelain or blue-white porcelain), a selection is made regarding the type and thickness of glazing, or extra coating using glazing is omitted.

Further, when the direction of the tip of the spout (3p) of the container (3) is designed to have specific angle θ, that is, 90°±15° (in particular, 90°±10°) downward from a horizontal plane, a working effect of not having adhesion of the viscous melted liquid as caused by cooling and solidification of the viscous melted liquid spilled over to a backside of the spout (3p), which occurs at the time of bringing the container back to an upright position after pouring, can be exhibited, which is particularly preferable.

In general, the spout angle of a common container is almost horizontal or tilted slightly in downward direction. As such, designing the direction of the tip of the spout (3p) of the container (3) to be almost in the right downward direction as described above enables a sophisticated feeling of ceramics (in particular, white porcelain or blue-white porcelain) and unique characteristics in terms of the design so that it is particularly preferable from the viewpoint of the purpose of the present invention.

It is also advantageous from the viewpoint of the purpose of the present invention that only the inner surface of the container (3) made of ceramics is prepared as a smooth surface according to glazing and the whole or most of the outer surface of the container (3) is left as a biscuit-fired surface.

As the inner surface is smooth, pouring of the composition (1) from the container (3) can be performed without a problem and also an effect of preventing an infiltration of an easily infiltrating component in the composition (1) to the inner surface of the container wall can be exhibited.

Meanwhile, as the outer surface is left as a biscuit-fired surface, stable impression is yielded as gloss or reflection is suppressed and also there is a merit that a slip-preventing effect is exhibited when the container (3) is carried on hands.

In addition, determining whether or not the inner surface or outer surface of the container (3) has a smooth surface can be made based on a difference in gloss, but it can be easily and clearly determined by pencil writing.

### (Hair-Care Product of Indirect Heating Type Using Fire)

This hair-care product is a hair-care product in which a composition present as a solid at room temperature is fluidized according to direct or indirect heating and melting by using fire.

This hair-care product of indirect heating type using fire consists of the composition (1) which is present as a solid at room temperature and contains theobroma grandiflorum seed butter as a main component, the container (3) for accommodating the composition (1), the heating means (4) for melting or fluidizing the composition (1) within the container (3) by heating externally the container (3) accommodating the composition (1) with fire, and the housing (5) with an incense burner shape for accommodating each of those members.

As the container (3) is heated by the heating means (4) and also the composition (1) added therein is melted and fluidized, an operation of applying the melted liquid on fingers or palm and transferring onto hair may be performed.

### BRIEF DESCRIPTION OF DRAWINGS

FIG. 1 is a side view illustrating an example of a candle type hair-care product among hair-care products of the present invention, in which a part of the product is expressed as a cut-out.

FIG. 2 is an exploded view illustrating diagrammatically an example of a hair-care product of indirect heating type using fire among the hair-care products of the present invention.

### MODE(S) FOR CARRYING OUT THE INVENTION

Hereinbelow, the present invention is explained in detail. Meanwhile, as significant explanations have been already given in “EFFECT OF THE INVENTION” described above, reference should be made to the descriptions in the section.

### (Hair-Care Product/Composition (1))

The hair-care product of the present invention is used as follows: a composition which is present as a solid at room temperature is heated and melted directly or indirectly by using fire, and the composition, thus fluidized, is applied to hair, in which the composition consists of the composition (1) which contains theobroma grandiflorum seed butter as a main component.

As described in the above “BACKGROUND ART”, theobroma grandiflorum seed butter is a fat having a water-holding property as obtained by adsorbing and purifying fats from seeds of cupuass fruit that is harvested in Amazon, and it is commercially available under the product name of “cropure cupuass” by Croda Japan KK.

Examples of the fats having a water-holding property which can be used in combination with theobroma grandiflorum seed butter include fats with a low melting point such as Arecaceae babassu oil, Anacardiaceae mango seed oil, or Dipterocarpaceae Shorea stenoptera butter.

Examples of the water-holding fats other than those described above include those described below.

Olive oil, camellia oil, almond oil, camellia japonica oil, safflower oil, sun flower oil, cotton seed oil, sesame oil, soy bean oil, corn oil, rapeseed oil, rice bran oil, castor oil, coconut oil, avocado oil, palm oil, annatto oil, linseed oil, jojoba oil, tea seed oil, peanut oil, avocado seed oil, hazel nut oil, rose hip oil, or the like.

Lanolin, bee wax, canderilla wax, carnauba wax, rice bran wax, rice wax, Japanese wax, tallow, cacao butter, hydrogenated tallow, shea butter, or the like.

As the composition (1) preferably has a melting point in the range of 35 to 65° C., waxes with a high melting point or wax-based water-holding fats are often blended in combination with theobroma grandiflorum seed butter or blended with oils to have a preparation which is a solid at room temperature and has a melting point in the range of 35 to 65° C.

The ratio of theobroma grandiflorum seed butter in the composition (1) is preferably high blending ratio like 60% by weight or more. More preferably, it is 70% by weight or more. Particularly preferred range is 80% by weight or more.

### (Other Additives)

Meanwhile, the composition (1) may contain various effective components and other additives that are related to hair care, as long as the spirit of the present invention is not impaired.

For example, branched fatty acids with carbon atom number of 14 to 28 and fatty acids with carbon atom number of 14 to 28 contribute to adhesion of both to hair cuticle (hair surface), and thus both are an effective component which is desirably blended. Lecithin is also an effective component for hair care, and as it is contained in a slight amount in theobroma grandiflorum seed butter, it can be blended complementally.

Examples of other additives include an anti-oxidant, an ultraviolet absorbing agent, a preservative, an anti-microbial agent, an anti-inflammatory agent, a refreshing agent, a pH controlling agent, pigments, fragrances, and hydrocarbon-based or oxyhydrocarbon-based wax.

### (Method for Use)

As it has been already described in “EFFECT OF THE INVENTION” described above, the hair-care product consisting of the composition (1) of the present invention has a method for use in which a melted liquid thereof is applied to fingers or palm and transferred onto hair.

### [Candle Type Hair-Care Product]

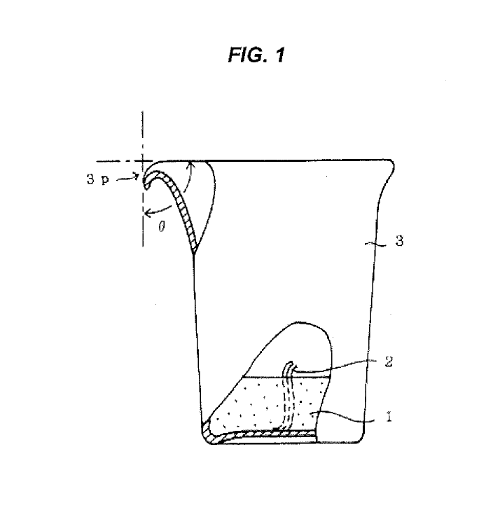

FIG. 1 is a side view illustrating one example of the candle type hair-care product among the hair-care products of the present invention, in which a part of the product is expressed as a cut-out.

The candle type hair-care product of the present invention consists of the composition (1) which is present as a solid at room temperature and contains theobroma grandiflorum seed butter as a main component, the wick (2) set in the composition (1), and the container (3) for accommodating the composition (1) attached with the wick (2). The composition (1) has been already explained in the above.

For using the candle type hair-care product, it is only required to light simply the wick (2) set in the composition (1). With the heat generated by lighting of the wick (2), the region surrounding the wick (2) in the container (3) is melted or fluidized, and thus the method for use includes applying the melted liquid on fingers or palm and transferring it onto hair.

### (Wick (2) and Container (3))

The candle type hair-care product has, as a constitutional element, the container (3) for accommodating the composition (1) and the wick (2) for melting the composition (1), in addition to the composition (1). Meanwhile, the composition (1) may be filled in the container (3) or prepared separately from the container (3).

In a commercial product, the wick (2) is generally set (installed) in the composition (1) which is filled in the container (3). When the composition (1) is sold in the form of a refill, it is preferable that the wick (2) is set in advance in the composition (1).

“Western style wick” consisting of cotton thread or the like is one example of the wick (2) which is universally used for a western candle (that is, western style candle).

The other example of the wick (2) is “Japanese style wick” used for a Japanese style candle. There are several types of a Japanese style wick depending on the production method, as exemplified below.

One of them is a Japanese style wick which is prepared by wrapping a winding bar with Japanese paper, wrapping up the resultant with several white cores collected from rush (Juncus effusus Linne) without any gap, and, if necessary, wrapping the resultant with thinly extended floss.

Another is a Japanese style wick in which only the Japanese paper is wrapped in hollow shape.

Still another is a Japanese style wick prepared by wrapping Japanese paper around an axis of barrel-shaped rush.

When a candle type hair-care product with a Japan-like image is to be produced, it is often preferable to use the “Japanese style wick” as the wick (2).

### (Container (3))

The material of the container (3) is arbitrarily chosen as long as it has inflammability, flame retardancy or heat resistance. However, considering that the candle type hair-care product of the present invention relates to a cosmetic, it is particularly preferably made of ceramics which is functionally excellent and also excellent in terms of the image.

In addition to the above, it is particularly preferable that the container (3) is a container with a deep bottom and it allows leak of light through a container wall at the time of lighting the wick (2).

Allowing the leak of light inside the container (3) through a container wall can be achieved by studying the materials of porcelain or figuring out the calcination method to have toughness even when the container wall is prepared to be thin.

Herein, the material of the container (3) made of ceramics is not particularly limited. However, preferred examples of the material include white porcelain and blue and white porcelain. That is because those made of white porcelain or blue and white porcelain have a sophisticated feeling, stability, and suitable weight feeling and thus are mostly suitable in view of the purpose of the present invention.

When the container (3) is made of ceramics, it is advantageous that the inner surface of the container (3) is prepared as a smooth surface according to glazing and the whole or most of the outer surface of the container (3) is left as a biscuit-fired rough surface. Determination of a smooth surface or a rough surface can be made based on a difference in gloss. However, as pencil writing is impossible on a smooth surface but it can be made on a rough surface, the determination can be easily and clearly made by pencil writing.

Further, as it has been discussed in “EFFECT OF THE INVENTION” described above, the container having a smooth inner surface poses no problem for the pouring property of the composition (1) and also an effect of preventing an infiltration of a specific component in the composition (1) to the container wall is exhibited. Meanwhile, as the outer surface is left as a biscuit-fired surface, stable impression is yielded and also there is a merit that a slip-preventing effect is exhibited when the container (3) is carried on hands.

(Spout (3p) of Container (3))

Further, as it has been discussed in “EFFECT OF THE INVENTION” described above, the direction of the tip of the spout (3p) of the container (3) is particularly preferably designed to have an angle of 90°±15° (in particular, 90°±10°) downward from a horizontal plan.

When it is formed at such angle 0, adhesion and solidification of the viscous melted liquid do not occur as caused by spilling over of the viscous melted liquid toward a backside of the spout (3p) after pouring the melted or fluidized composition (1), which is particularly preferable.

The angle of the spout (3p) of a common container is almost horizontal or tilted slightly in downward direction. As such, designing the direction of the tip of the spout (3p) of the container (3) to be almost in the right downward direction enables ceramics to have unique characteristics in terms of the design in addition to a sophisticated feeling of ceramics (in particular, white porcelain or blue-white porcelain).

### (Covering Body of Container (3))

Although it has not been illustrated, it is preferable that the container (3) is equipped with a covering body.

The material of the covering body can be any type, but it is particularly preferable that it be made of a material like wood, bamboo, vine, ivy, or the like.

The covering body plays a role of extinguishing fire, holding the container (3), and giving extra value to the product.

In the candle type hair-care product, when the wick (2) is lighted inside the container (3), fire of the wick (2) can be quickly extinguished by placing back the covering body on the opening of the container (3). In that case, there is often a space present at the spout (3p) of the container (3), but the fire can be quickly extinguished even in such case, because the introduction of air to the container (3) is limited.

At the time of use or during use of the candle type hair-care product, the covering body can be utilized as a holder for insulation purpose.

Furthermore, when the covering body is employed as a holder in the candle type hair-care product, it is also desirable from the viewpoint of interior design, and thus the value as a commercial product is also increased.

### [Hair-Care Product of Indirect Heating Type Using Fire]

This type of a hair-care product consists of the composition (1) which is present as a solid at room temperature and contains theobroma grandiflorum seed butter as a main component, the container (3) for accommodating the composition (1), the heating means (4) for melting or fluidizing the composition (1) within the container (3) by heating externally the container (3) accommodating the composition (1) with fire, and the housing (5) with an incense burner shape for accommodating each of those members.

As the housing (5) is often prepared to have a size which is the same as the size of a common tea incense-burner, the container (3) naturally becomes small, and thus the amount of the composition (1) to be accommodated in the container (3) becomes also small. Meanwhile, the container (3) is often used such that it is placed like a cover on top of the housing (5).

Structure of the container (3) or the spout (3p) thereof can be the same as that of the candle type hair-care product. However, it is often prepared to have a simple shape like a liquor glass shape (that is, shape of a small dish). Because the container (3) is small, the spout (3p) may be either formed or not formed.

As for the heating means (4), various kinds can be used as long as it can heat, from the lower side, the bottom of the container (3) by using fire. Representative examples of the heating means (4) include a means of using a candle; a means of lighting charcoal, alcohol in solid form, ash for pocket heater, or the like; a means with a cartridge using liquefied gas; and a means of using an alcohol lamp. Other heating means may include a means of heating using a double boiler like performing the heating by immersion in hot water boiled by using fire.

### EXAMPLES

Hereinbelow, the present invention is further explained in view of Examples.

### Example 1

### Formulation of Composition (1)

By kneading a mixture with the following formulation under heating at the temperature which is equal to or higher than its melting temperature, the composition (1) for Example 1 was prepared. The melting point of the composition was 50° C. or so.

### (Formulation)

Theobroma grandiflorum seed butter . . . 86.97% by weight (Note)

Olive oil . . . 7.00% by weight

Japanese wax . . . 5.00% by weight

Lanoiln . . . 1.00% by weight

Mixture of fatty acids with carbon number of 14 to 28 . . . 0 to 1.00% by weight (Note)

Tocopherol . . . 0.03% by weight

### (Note)

The ratio c of the fatty acids with a carbon number of 14 to 28 varies within the range of 0 to 1.00% by weight as described below. In the formulation shown above, c is 0% by weight. Because the adjustment of the formulation is basically performed based on the ratio of theobroma grandiflorum seed butter, when the ratio c is 1.00% by weight, the ratio of theobroma grandiflorum seed butter is reduced by 1.00% by weight from the formulation shown above.

Major role of each component in the above formulation is as described below.

Theobroma grandiflorum seed butter is a main component which constitutes most of the composition, and it plays an important and critical role for hair care.

Olive oil is an oil having a water-holding property. Meanwhile, according to the present formulation in which theobroma grandiflorum seed butter is blended in a high amount, the composition becomes excessively hard. Thus, it plays a role of providing flexibility by reducing the hardness.

Lanolin is a wax having a water-holding property, and it is a component for controlling the degree of hardness and softness and the feeling at the time of use.

Fatty acids with hydrocarbon number of 14 to 28 are a component for enhancing tidy feel of hair.

Tocopherol (vitamin E) is a component which plays a role of an anti-oxidant.

### (Preparation of Container (3))

Several container (3) having a deep bottom as shown in FIG. 1 were prepared. The container was made of white porcelain. The production was achieved by requesting a professional potter in Kyoto Kiyomizu.

The container (3) has a size which is one-level bigger than a paper cup. Height of the container (3) is 95 mm, diameter of the upper surface is 75 mm, and diameter of the lower surface is 57 mm. Thickness is 2 mm.

Because the inner surface of the container (3) is finished smoothly by glazing, pencil writing was impossible. This means that there is no infiltration of a specific component of the composition (1) to the inner surface of a container wall.

Meanwhile, because the outer surface of the container (3) is left as a biscuit-fire surface, it was easy to perform pencil writing. This means that the container (3) does not easily slip and fall when carried on hands.

Navy-colored patterns are drawn on the outer surface of the container (3). However, expression of the patterns is omitted in FIG. 1.

(Spout (3p))

As shown in FIG. 1, the spout (3p) is formed in the container (3). The direction of the tip of the spout (3p) is almost the right downward direction (that is, it is formed almost at 90° downward from the horizontal plane).

For such reasons, a situation of adhesion and solidification of a melted liquid as the solution is spilled over to the backside of the spout (3p), which happens at the time of pouring the viscous melted liquid as the contents flow from the spout (3p), does not occur.

### (Candle Type Hair-Care Product)

The composition (1) which has been prepared above was heated again for fluidization. Then, it was poured into the container (3) and filled up to 30 mm point from the internal bottom. It was subsequently cooled to a room temperature.

Meanwhile, while the composition (1) poured in the container (3) maintains the fluidity, the wick (2) made of threads which have been immersed in advance in the composition (1) followed by winding was embedded and set in the center position of the composition (1). Meanwhile, the material of the wound threads was a cotton thread.

Meanwhile, the composition (1) having a shape which is tightly fitted in the container (3) and the wick (2) attached thereto was prepared separately and then set in the container (3). As a result, the same candle type hair-care product as above was obtained.

When the wick (2) in the container (3) is lighted, light is leaked through a container wall of the container (3), yielding a fantastic atmosphere.

Meanwhile, since a long-stem match with stem length of 10 cm or so is commercially available, the lighting of the wick (2) in the container (3) is preferably performed by using a long-stem match. That is because use of a match goes well with a place in which the hair care is performed.

### Examples 1A to 1D

Samples having the above formulation but the ratio of the fatty acids with a carbon number of 14 to 28 are varied in the range of 0 to 1.00% by weight were prepared.
Example 1A: Fatty acids with a carbon number of 14 to 28 are not blended (blend amount of 0%)
Example 1B: Fatty acids with a carbon number of 14 to 28 are blended at 0.05% by weight
Example 1C: Fatty acids with a carbon number of 14 to 28 are blended at 0.50% by weight
Example 1D: Fatty acids with a carbon number of 14 to 28 are blended at 1.00% by weight

### (Samples 1 to 3 of Comparative Example)

The following samples 1 to 3, which are a well-known commercially available treatment agents (all of the components are publicly known also in the internet), were prepared.
Sample 1: Essence for repair of damaged hair, which is commercially provided by company A
Sample 2: Conditioner for care of damaged hair, which is commercially provided by company B
Sample 3: Conditioner for damaged hair, which is commercially provided by company C

### (Evaluation Method/Preparation of Human Hair)

By using commercially available processed white human hair (that is, product after bleaching treatment), several locks of hair, each having weight of 2 g and length of 25 cm, were prepared. Subsequently, the lock of hair was washed using shampoo, briefly rinsed, and air-dried to prepare a lock of hair for test.

### (Test Method)

The lock of hair for test which has been prepared in the above was coated with a sufficient amount of the treatment of the sample 1 to 3 of Comparative Example described above followed by rinsing with warm water at 40° C. After that, it was towel-dried and additionally air-dried.

Furthermore, the lock of hair for test which has been prepared in the above was applied with the sample of Examples 1A to 1D described above. In this case, the composition (1) inside the container (3) was melted and fluidized by lighting the wick (2), and then coated on the lock of hair for test prepared in the above, followed by natural cooling.

### (Test Results)

The mess width was measured for the lock of hair obtained after the treatment and the “tidy hair feel” was determined based on the degree of the mess width.

—Commercially Available Treatment—

Sample 1 . . . mess width of 8 cm

Sample 2 . . . mess width of 7 cm

Sample 3 . . . mess width of 6 cm

### Examples 1A to 1D

Example 1A . . . mess width of 3 cm
Example 1B . . . mess width of 3 cm
Example 1C . . . mess width of 2 cm
Example 1D . . . mess width of 2 cm

### —Discussions—

As described above, the mess width at the time of using a commercially available treatment is believed to be the level which indicates the most up-to-date technique of a current treatment method.

Since the “application means” is different, no direct comparison can be made between Examples 1A to 1D and the treatment method. However, in terms of the “tidy hair feel”, it was found to yield a desirable result.

Namely, it can be said that the candle type hair-care product using the composition (1) in which theobroma grandiflorum seed butter is contained as a main component (about 86 to 87% by weight in Examples 1A to 1D) (that is, a hair-care product which is applied to hair after fluidization by direct heating and melting by using fire) is a product belonging to the category which is different from that of a conventional hair-care product (treatment).

It was also found that, with consideration of the use or the use ratio of the fatty acids with the carbon number of 14 to 28 in the candle type hair-care product, more preferred results are obtained like Examples 1C or 1D of the above test results.

### Comparative Example 1

The ratio of theobroma grandiflorum seed butter was reduced to “50% by weight”, “40% by weight”, “30% by weight”, “10% by weight”, or “5% by weight” in the formulation of the composition (1) of Example 1, and the reduced amount was replaced with various fats, waxes, and oils for further test.

However, having a candle type itself was difficult or not possible to achieve, or even when a candle type itself can be prepared, only the result indicating that the hair care performance is clearly worse than Example 1 above was obtained.

### Example 2

As the wick (2), Japanese style wick A having an outer diameter of about 5 mm was prepared by wrapping a winding bar with Japanese paper according to barrel winding (body winding), wrapping up the resultant with 3 or 4 white cores collected from a stem of rush (Juncus effusus var. decipens) without any gap, additionally wrapping the resultant with thinly extended floss silk, and removing the wrapped laminate from the winding bar.

As the wick (2), Japanese style wick B having an outer diameter of about 5 mm was also prepared by wrapping a hollow body of a stem of rush from which cores are removed with Japanese paper according to barrel winding (body winding).

Two kinds of candle type hair-care products were prepared in the same manner as Example 1 except that each of Japanese style wick A and Japanese style wick B prepared in the above is used instead of the wick (2) “made of threads which have been immersed in advance in the composition (1).” Meanwhile, these Japanese style wick A and Japanese style wick B were also immersed in advance in the composition (1).

When the wick (2) in the container (3) is lighted, the atmosphere created by this hair-care product exhibits a Japanese-style impression (image) compared to Example 1.

Further, this hair-care product did not cause a state in which the wick (2) burn-out first while the composition (1) is still present (that is, a state in which the composition (1) is wasted).

### Example 3

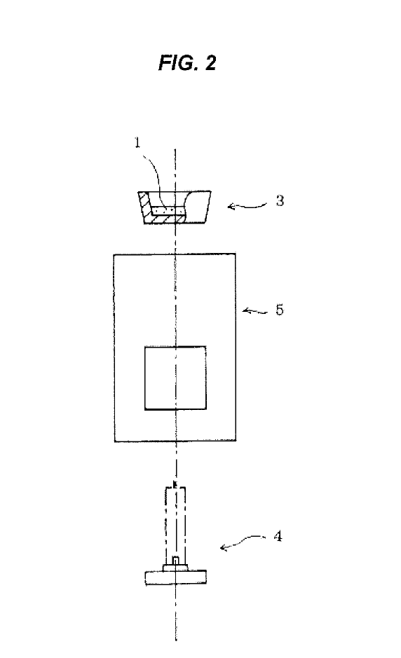

FIG. 2 is an exploded view illustrating diagrammatically one example of the hair-care product of indirect heating type using fire among the hair-care products of the present invention.

A set comparable to a commercially available tea incense-burner, that is, a set consisting of the housing (5) with an incense-burner shape which has a bottom, an approximately barrel shape, and a window on a side wall, the heating means (4) consisting of a candle and a dish for accommodating it, and the container (3) with a small dish shape (in the case of tea incense-burner, tea leaves are contained therein) was prepared. The container (3) with a small dish shape is used in such a manner that it is applied over the opening on top of the housing (5) with barrel shape.

To the container (3) with a small dish shape, the composition (1) in solid form with the formulation described in the above Example 1C was added, and the candle was lighted. When the composition (1) is melted and fluidized, the fire is extinguished if necessary, and the melted liquid within the container (3) with a small dish shape was applied on fingers and palm and then transferred onto hair.

### INDUSTRIAL APPLICABILITY

The hair-care product of the present invention has a special method for use as follows: a composition present as a solid at room temperature is heated and melted directly or indirectly by using fire, and the composition, thus fluidized, is applied to hair. Further, by exhibiting a desirable working effect, it is useful as a product for hair care.

### EXPLANATIONS OF LETTERS OR NUMERALS

(1) . . . Composition
(2) . . . Wick
(3) . . . Container
(3p) . . . Spout
(4) . . . Heating means
(5) . . . Housing

1. A hair-care product characterized in that;
it is a hair-care product which is used as follows: a composition present as a solid at room temperature is heated and melted directly or indirectly by using fire, and the composition, thus fluidized, is applied to hair, and
the composition comprises a composition (1) which contains theobroma grandiflorum seed butter as a main component.

2. The hair-care product according to claim 1, wherein a ratio of theobroma grandiflorum seed butter in the composition (1) is 60% by weight or more.

3. A candle type hair-care product characterized in that;
it comprises a composition (1) which is present as a solid at room temperature and contains theobroma grandiflorum seed butter as a main component, a wick (2) set in the composition (1), and a container (3) for accommodating the composition (1) attached with the wick (2), and
the composition (1) attached with the wick (2) is either filled or fillable in the container (3) and a region surrounding the wick (2) in the container (3) is melted and fluidized by heat generated at the time of lighting the wick (2).

4. The candle type hair-care product according to claim 3, wherein the container (3) is made of ceramics and has a deep bottom, and light is leaked to an outside through a container wall at the time of lighting.

5. The candle type hair-care product according to claim 3, wherein a direction of the tip of a spout (3p) of the container (3) is formed at an angle θ of 90°±15° downward from a horizontal plane.

6. The candle type hair-care product according to claim 3, wherein the wick (2) comprises a hollow core in which Japanese paper or Japanese paper and rush (Juncus effusus Linne) is used as a core material.

7. A hair-care product of indirect heating type using fire characterized in that it comprises:
a composition (1) which is present as a solid at room temperature and contains theobroma grandiflorum seed butter as a main component,
a container (3) for accommodating the composition (1),
a heating means (4) for melting or fluidizing the composition (1) within the container (3) by heating externally the container (3) accommodating the composition (1) with fire, and
a housing (5) with an incense burner shape for accommodating each of those members.
